(19) United States
(12) Patent Application Publication
(10) Pub. No.: US 2015/0177246 A1
(43) Pub. Date: Jun. 25, 2015

(54) FUSION GENE OF CEP55 GENE AND RET GENE

(71) Applicants: NATIONAL CANCER CENTER, Tokyo, JP (JP); LSIP, LLC, Tokyo, JP (JP)

(72) Inventors: Tatsuhiro Shibata, Tokyo (JP); Fumie Hosoda, Tokyo (JP)

(73) Assignees: LSIP, LLC, Tokyo, JP (JP); NATIONAL CANER CENTER, Tokyo, JP (JP)

(21) Appl. No.: 14/417,052

(22) PCT Filed: Jul. 23, 2013
(86) PCT No.: PCT/JP2013/069929

§ 371 Date: Jan. 23, 2015

(30) Foreign Application Priority Data
Jul. 26, 2012 (JP) 2012-165694

(51) Int. Cl. G01N 33/574; C07K 16/32; C07K 14/82; C12Q 1/68; C12N 9/12

(52) U.S. Cl. CPC G01N 33/5748; C12Q 1/6886; C12N 9/12; C07K 14/82; C07K 16/32; C12Q 2600/106; C07K 2319/00; C12Y 207/10001; C12Q 2600/158

(57) ABSTRACT

In order to identify genes that can serve as indicators for predicting the effectiveness of drug treatments in cancers and provide novel methods for predicting the effectiveness of treatments with drugs targeting said genes, transcriptome sequencing was performed of diffuse-type gastric cancer. As a result, in-frame fusion transcripts between the CEP55 gene and the RET gene were identified. It was also found that said gene fusions induce activation of RET protein, thereby causing canceration of cells. Further, it was demonstrated that the RET protein activation and canceration caused by said gene fusion can be suppressed by using a RET tyrosine kinase inhibitor, and that treatments with a RET tyrosine kinase inhibitor are effective in patients with detection of said gene fusion.

### TECHNICAL FIELD

The present invention relates to fusion genes between the CEP55 gene and the RET gene, and more particularly to polynucleotides encoding fusion polypeptides between CEP55 protein or part thereof and RET protein or part thereof, polypeptides encoded by said polynucleotides, and a method for detecting said polynucleotides or polypeptides. This invention also relates to a method for determining the effectiveness of cancer treatments with a RET tyrosine kinase inhibitor targeting said polynucleotides or polypeptides. This invention further relates to a method for cancer treatment using said effectiveness determination. Furthermore, this invention relates to agents for use in these methods.

### BACKGROUND ART

Gastric cancer is a cancer of which the second largest number of people in the world die, and is one of cancers from which many patients suffer in East Asia. The prognosis of this cancer has improved particularly in Japan due to the progress in early diagnostic technologies including endoscopy. However, patients with advanced or recurrent gastric cancer are still difficult to treat, and the 5-year survival rate of patients with this cancer in many countries including Europe and the United States is not more than 30%.

Gastric cancer is histopathologically classified into the following two major types: intestinal-type and diffuse-type (according to Lauren classification). The latter type of cancer, which is common in young people, easily develops peritoneal dissemination or lymph node metastasis, and has poor prognosis. The principal method adopted for treating gastric cancer is surgical operation, but patients treated with this method tend to easily develop peritoneal dissemination or metastases to other organs, so the diffuse-type gastric cancer associated with a high recurrence rate is difficult to treat with surgical operation. In addition, there has been developed no anticancer drug effective against gastric cancer, including diffuse-type cancer. Thus, to date, no therapeutic method has been established which is effective for gastric cancer, in particular diffuse-type gastric cancer.

Regarding gastric cancer, there have been reports on amplification of the EGFR, FGFR2, MET, ERBB2 and MYC genes, mutation of the KRAS, TP53 and CDH1 genes, and other alternations. At present, various molecular targeted therapies for gastric cancer have been studied which target the EGFR, FGFR2, ERBB2 or other gene. Thus, in the field of various cancers including gastric cancer, there is a strong demand for identifying oncogenes involved in the onsets of such cancers, such as mutant genes (mutant proteins) and fusion genes (fusion proteins), because such an identification will greatly contribute to development of novel cancer treatment and testing methods targeting such genes.

As regards other cancers, it has been reported that activating mutations of the RET gene occur in familial and sporadic thyroid cancer, some cases of colon cancer, and pheochromocytoma. It was also found that the RET gene is a responsible gene for multiple endocrine neoplasia (MEN) syndrome (MEN2A, MEN2B) (Non-patent Document 1). Further, the PTC-RET fusion gene was reported as a genomic aberration characteristic of thyroid cancer (Non-patent Document 2), and the presence of this type of gene was recently observed in lung cancer as well (Non-patent Document 3). Furthermore, the presence of the KIF5B-RET fusion gene has been found as a genomic aberration in lung adenocarcinoma (Non-patent Documents 3-5).

As low-molecular-weight inhibitors for the RET gene, vandatinib, motesanib, sorafenib, sunitinib, XL-184 and the like are known, and some of them are now being clinically developed as anticancer drugs.

In addition, various studies are made regarding using the foregoing mutant and fusion genes as indicators for predicting the effectiveness of treatments of cancers with said inhibitors. For example, it has been shown that tyrosine kinase inhibitors targeting EGFR or ALK protein are particularly effective for the treatment of lung adenocarcinoma harboring EGFR mutations and/or ALK fusions. Further, a technique for detecting a fusion of the ALK tyrosine kinase gene as observed in 4-5% of lung cancer cases was developed and clinically tested as a method to screen for cases indicated for inhibitors against ALK protein tyrosine kinase.

On the other hand, the CEP55 gene is found to be a microtubule-associated molecule which plays an important role in cytokinesis (Non-patent Document 6). This protein has multiple coiled coil domains including N-terminal region, and is presumed to be capable of dimerizing. Also, CEP55 protein is highly expressed in a wide variety of cancer cells, but is expressed at a low level in normal tissues, except that it is highly expressed in normal testis. Thus, CEP55 protein is known as a so-called cancer-testis antigen and is studied as a target for cancer vaccine therapies (Non-patent Document 7).

However, a thorough elucidation of fusion genes and other genes in various cancers including gastric cancer has not yet been achieved, and there is at present a demand for identifying fusion genes and other genes which contribute greatly to development of novel cancer treatment and testing methods and can also serve as indicators for predicting the effectiveness of drug treatments.

### CITATION LIST

### Non-Patent Documents

Non-patent Document 1: Kouvaraki M A., et al., Thyroid, 2005, vol. 15, p. 531-544
Non-patent Document 2: Nikiforov Y E., et al., Nat Rev Endocrinol., 2011, vol. 7, p. 569-580
Non-patent Document 3: Takeuchi K., et al., Nat Med., 2012, vol. 18, p. 378-381
Non-patent Document 4: Kohno T., et al., Nat Med., 2012, vol. 18, 375-377
Non-patent Document 5: Lipson D., et al., Nat Med., 2012, vol. 18, p. 382-384
Non-patent Document 6: Zhao W M., et al., Mol Biol Cell., 2006, vol. 17, p. 3881-3896
Non-patent Document 7: Inoda S., et al., J Immunother., 2009, vol. 32, p. 474-485

### SUMMARY OF INVENTION

### Technical Problem

The present invention has been made in consideration of the above-described problems with the prior art, and has as its object to identify genes that can serve as indicators for predicting the effectiveness of drug treatments in gastric cancer and other cancers. Another object of this invention is to provide novel methods for predicting the effectiveness of drug treatments targeting said genes and expression products thereof. Still another object of this invention is to provide methods for treating gastric cancer and other cancers on the basis of the prediction of the effectiveness of drug treatments targeting said genes and expression products thereof. Yet another object of this invention is to provide agents for use in detecting said genes and expression products thereof in these methods.

### Solution to Problem

As a result of intensive studies to achieve the above-mentioned objects, the present inventors have identified in-frame fusion transcripts between the CEP55 gene and the RET gene by performing transcriptome sequencing of 13 diffuse-type gastric cancer (DGC) specimens. Thus, it is considered that the fusion gene between the CEP55 gene and the RET gene (CEP55-RET fusion gene) is generated by reciprocal translocation between two human chromosomes 10 or by breakage and reunion in human chromosome 10. In fact, the inventors have investigated the nucleotide sequences of genomic fusion sites, and as a result, have found cases of fusion generated by breakage and reunion in human chromosome 10.

Thus, the inventors have introduced the CEP55-RET gene into normal cells, and have observed that the cells acquire anchorage-independent colony-forming ability, in other words become cancerous. We also have found that in these cells, the RET protein kinase is activated and also phosphorylation of its downstream signals such as AKT is increased.

Further, the inventors have subcutaneously transplanted CEP55-RET fusion gene-expressing cells into nude mice and, as a result, have observed that said cells have in vivo tumorigenic ability.

On the other hand, it has also been demonstrated that the activation of RET protein kinase, the phosphorylation of AKT and the like, the anchorage-independent colony-forming ability, and the in vivo tumorigenic ability are significantly suppressed by using a RET tyrosine kinase inhibitor or through inactivation of the kinase domain of the CEP55-RET fusion polypeptide.

On the basis of the above-described findings, the present inventors have found that it is possible to predict the effectiveness of treatment with a drug targeting this gene fusion in diffuse-type gastric cancer and other cancers, and that efficient treatments can be achieved by administering the drug to patients in whom the treatments with the drug have been determined to be effective on the basis of this prediction; thus, the inventors have completed the present invention.

Therefore, the present invention relates to polynucleotides encoding fusion polypeptides between CEP55 protein or part thereof and RET protein or part thereof, polypeptides encoded by said polynucleotides, a method for detecting said polynucleotides or polypeptides, a method for determining the effectiveness of cancer treatments with a RET tyrosine kinase inhibitor using the presence of said polynucleotides or polypeptides as an indicator, a method for treatment of cancer utilizing said effectiveness determination, and agents for use in these methods. More specifically, this invention provides the following:

[1] A polynucleotide encoding a polypeptide in which CEP55 protein or part thereof and RET protein or part thereof are fused together.

[2] A polypeptide encoded by the polynucleotide as set forth in [1].

[3] A method for detecting the presence or absence in a sample of the polynucleotide as set forth in [1] or of the polypeptide as set forth in [2], the method comprising the steps of:
(a) contacting the sample with an agent intended for specifically detecting the presence or absence of the polynucleotide or the polypeptide in the sample; and
(b) detecting the presence or absence of the polynucleotide or the polypeptide.
[4] An agent for detecting the presence or absence in a sample of the polynucleotide as set forth in [1] or of the polypeptide as set forth in [2] by the method as set forth in [3], the agent comprising a polynucleotide or polynucleotides as set forth below in any one of (a) to (c), the polynucleotide or polynucleotides having a chain length of at least 15 nucleotides, or an antibody as set forth below in (d):

(a) a polynucleotide or polynucleotides that are at least one probe selected from the group consisting of a probe that hybridizes to a polynucleotide encoding CEP55 protein and a probe that hybridizes to a polynucleotide encoding RET protein;

(b) a polynucleotide that is a probe that hybridizes to a point of fusion between a polynucleotide encoding CEP55 protein and a polynucleotide encoding RET protein;

(c) polynucleotides that are a pair of primers designed to sandwich a point of fusion between a polynucleotide encoding CEP55 protein and a polynucleotide encoding RET protein; and

(d) an antibody that binds to a polypeptide in which CEP55 protein and RET protein are fused together.

[5] A method for determining the effectiveness of a cancer treatment with a RET tyrosine kinase inhibitor, the method comprising the step of detecting the presence or absence in a sample isolated from a patient of the polynucleotide as set forth in [1] or of the polypeptide as set forth in [2], wherein in a case where the presence of the polynucleotide or the polypeptide is detected, the cancer treatment with the RET inhibitor is determined to be highly effective in the patient.
[6] An agent for determining the effectiveness of a cancer treatment with a RET inhibitor by the method as set forth in [5], the agent comprising a polynucleotide or polynucleotides as set forth below in any one of (a) to (c), the polynucleotide or polynucleotides having a chain length of at least 15 nucleotides, or an antibody as set forth below in (d):

(a) a polynucleotide or polynucleotides that are at least one probe selected from the group consisting of a probe that hybridizes to a polynucleotide encoding CEP55 protein and a probe that hybridizes to a polynucleotide encoding RET protein;

(b) a polynucleotide that is a probe that hybridizes to a point of fusion between a polynucleotide encoding CEP55 protein and a polynucleotide encoding RET protein;

(c) polynucleotides that are a pair of primers designed to sandwich a point of fusion between a polynucleotide encoding CEP55 protein and a polynucleotide encoding RET protein; and

(d) an antibody that binds to a polypeptide in which CEP55 protein and RET protein are fused together.

[7] A method for treatment of cancer, comprising the step of administering a RET tyrosine kinase inhibitor to a patient in whom a cancer treatment with the RET tyrosine kinase inhibitor has been determined to be highly effective by the method as set forth in [5].
[8] A therapeutic agent for cancer, comprising a RET tyrosine kinase inhibitor as an active ingredient, wherein the therapeutic agent is to be administered to a patient in whom a cancer treatment with the RET tyrosine kinase inhibitor has been determined to be highly effective by the method as set forth in [5].

### Advantageous Effects of Invention

The present invention enables effective detection of fusion genes between the CEP55 gene and the RET gene, and expression products thereof. This invention also makes it possible to predict the effectiveness of various treatments on cancers, in particular the effectiveness of cancer treatments with a RET tyrosine kinase inhibitor, on the basis of the detection of said fusion genes and expression products thereof. This prediction makes it possible to avoid administration of a drug to cancer patients conceivably not responsive to the administration of the drug, thereby allowing efficient cancer treatments.

### BRIEF DESCRIPTION OF DRAWINGS

FIG. 1 is a schematic diagram showing modes of the fusion between the CEP55 gene and the RET gene. The left half of this figure shows one mode of the CEP55-RET fusion, which is generated by reciprocal translocation between two human chromosomes 10, and the right half of this figure shows another mode of fusion which is generated by breakage and reunion in human chromosome 10.

FIG. 2 is a schematic diagram showing the fusion between CEP55 protein and RET protein. More specifically, this schematic diagram shows that the N-terminal moiety of CEP55 protein is fused with the C-terminal moiety of RET protein, which comprises a tyrosine kinase domain. This diagram also shows that a polynucleotide encoding a fusion polypeptide between CEP55 protein and RET protein can be detected by using RT-PCR primers each hybridizing to exon 2 in the CEP55 gene or exon 14 in the RET gene.

FIG. 3 is a schematic diagram showing a method for detecting the inventive fusion gene by the ISH method. More specifically, this schematic diagram shows that in the case of the CEP55-RET fusion gene, the presence of the CEP55-RET fusion gene generated by reciprocal translocation between two human chromosomes 10 can be detected by designing ISH probes each specific for a portion toward the 5′ end of the CEP55 gene or for a portion toward the 3′ end of the RET gene. In this figure, the black circle represents a probe hybridizing to a portion toward the 5′ end of the CEP55 gene, and the white circle represents a probe hybridizing to a portion toward the 3′ end of the RET gene (also in FIG. 4, probes are represented in the same way).

FIG. 4 is a schematic diagram showing a method for detecting the inventive fusion gene by the ISH method. More specifically, this schematic diagram shows that in the case of the CEP55-RET fusion gene, the presence of the CEP55-RET fusion gene generated by breakage and reunion in human chromosome 10 can be detected by designing ISH probes each specific for a portion toward the 5′ end of the CEP55 gene or for a portion toward the 3′ end of the RET gene.

FIG. 5 is a set of photos showing the results of observing, under a microscope, the anchorage-independent colony formation in normal murine fibroblast lines each stably expressing the wild-type or the mutated RET fusion polypeptide, which were each seeded in a soft agar medium. In this figure, the “CEP55-RET” panel shows the result for a cell line expressing the wild-type CEP55-RET fusion polypeptide, the “CEP55-RET-KD” panel shows the result for a cell line expressing the mutated CEP55-RET fusion polypeptide, the “KIF5B-RET” panel shows the result for a cell line expressing the wild-type KIF5B-RET fusion polypeptide, and the “KIF5B-RET-KD” panel shows the result for a cell line expressing the mutated KIF5B-RET fusion polypeptide. The bars in these panels indicate the length of 100 μm.

FIG. 6 is a graph showing the results of analyzing the anchorage-independent colony-forming ability of normal murine fibroblast lines stably expressing any one of the wild-type RET fusion polypeptides, which were each seeded in a soft agar supplemented with a RET tyrosine kinase inhibitor (vandetanib or XL184). In this graph, “CEP55-RET” represents the results for cell lines expressing the wild-type CEP55-RET fusion polypeptide, and “KIF5B-RET” represents the results for cell lines expressing the wild-type KIF5B-RET fusion polypeptide. The “VD” bars show the results of colony formation in the presence of vandetanib (at a vandetanib concentration in medium of 0.2 μM), the “XL” bars show the results of colony formation in the presence of XL184 (at an XL184 concentration in medium of 0.2 μM), and the “Blank” bars show the results of colony formation in the absence of a RET tyrosine kinase inhibitor. The vertical axis represents relative values calculated with respect to the number of colonies formed in each of the cell lines expressing any one of the wild-type RET fusion polypeptides in the absence of a RET tyrosine kinase inhibitor, which is taken as 100.

FIG. 7 is a set of photos showing the results of analyzing by Western blotting the phosphorylation of various proteins in normal murine fibroblast lines stably expressing any one of the RET fusion polypeptides, which were subjected to serum starvation and then cultured in the presence of a RET tyrosine kinase inhibitor (vandetanib or XL184). In this figure, “CEP55-RET” represents the results for cell lines expressing the CEP55-RET fusion polypeptide, and “KIF5B-RET” represents the results for cell lines expressing the KIF5B-RET fusion polypeptide. The lanes labeled as “W” show the results of culturing the wild-type RET fusion polypeptide-expressing cell lines in the absence of a RET tyrosine kinase inhibitor; the lanes labeled as “V” show the results of culturing the wild-type RET fusion polypeptide-expressing cell lines in the presence of vandetanib (at a vandetanib concentration in medium of 1 μM); the lanes labeled as “X” show the results of culturing the wild-type RET fusion polypeptide-expressing cell lines in the presence of XL184 (at a XL184 concentration in medium of 1 μM); and the lanes labeled as “KD” show the results of culturing the mutated RET fusion polypeptide-expressing cell lines in the absence of a RET tyrosine kinase inhibitor. Since these fusion polypeptides were further tagged with the FLAG tag and expressed intracellularly, the “FLAG tag” row in this figure shows the results of detecting the respective fusion polypeptides through this tag. The “p-RET (Y1062)” row shows the results of detecting phosphorylated RET. The “STAT3” and “p-STAT3 (Y705)” rows show the results of detecting STAT3 and phosphorylated STAT3, respectively. The “AKT” and “p-AKT (S473)” show the results of detecting AKT and phosphorylated AKT, respectively. The “MAPK” and “p-MAPK (T202/Y204)” rows show the results of detecting MAPK and phosphorylated MAPK, respectively. The “β-actin” row shows that the amount of protein used as an internal standard is uniform among the lanes.

FIG. 8 is a set of photos showing the results of subcutaneously transplanting each of normal murine fibroblast lines expressing any of the RET fusion polypeptides into immunodeficient mice. In this figure, the “CEP55-RET” panel is a photo taken upon the observation of an immunodeficient mouse 18 days after it was transplanted subcutaneously with the cell line expressing the wild-type CEP55-RET fusion polypeptide. The “KIF5B-RET” panel is a photo taken upon the observation of an immunodeficient mouse 18 days after it was transplanted subcutaneously with the cell line expressing the wild-type KIF5B-RET fusion polypeptide. Triangles indicate formed tumors. “8/8” represents that the cells of interest were transplanted into two sites of each of four mice, a total of eight sites, and as a result, tumorigenesis was observed in all of the eight sites. The “CEP55-RET-KD” panel is a photo taken upon the observation of an immunodeficient mouse 18 days after it was transplanted subcutaneously with the cell line expressing the mutated CEP55-RET fusion polypeptide. “0/6” represents that the cells of interest were transplanted into two sites of each of three mice, a total of six sites, and as a result, no tumorigenesis was observed in any of the six sites.

### DESCRIPTION OF EMBODIMENTS

### Polynucleotides Encoding Fusion Polypeptides Between CEP55 Protein and RET Protein, and Polypeptides Encoded by the Polynucleotides

As disclosed below in Examples, multiple cases of fusion between the CEP55 gene and the RET gene were first discovered according to the present invention. Therefore, this invention provides a polynucleotide encoding a fusion polypeptide between CEP55 protein or part thereof and RET protein or part thereof (said polynucleotide and said fusion polypeptide are to be hereinafter also referred to as the “CEP55-RET fusion polynucleotide” and the “CEP55-RET fusion polypeptide”, respectively).

The “CEP55-RET fusion polypeptide” as referred to in the present invention means a polypeptide in which the full length or part of CEP55 protein is fused with the full length or part of RET protein. Also, the “CEP55-RET fusion polynucleotide” as referred to in this invention means a polynucleotide in which a polynucleotide encoding the full length or part of CEP55 protein is fused with a polynucleotide encoding the full length or part of RET protein.

The “CEP55 (centrosomal protein 55 kDa) protein” according to the present invention refers to a protein encoded by the gene located at a long arm of chromosome 10 (10q23.3) in humans. In this invention, the “CEP55 protein”, as far as it is derived from humans, is typified by the protein consisting of the amino acid sequence of SEQ ID NO: 2. The polynucleotide encoding the CEP55 protein is typified by the polynucleotide consisting of the nucleotide sequence of SEQ ID NO: 1.

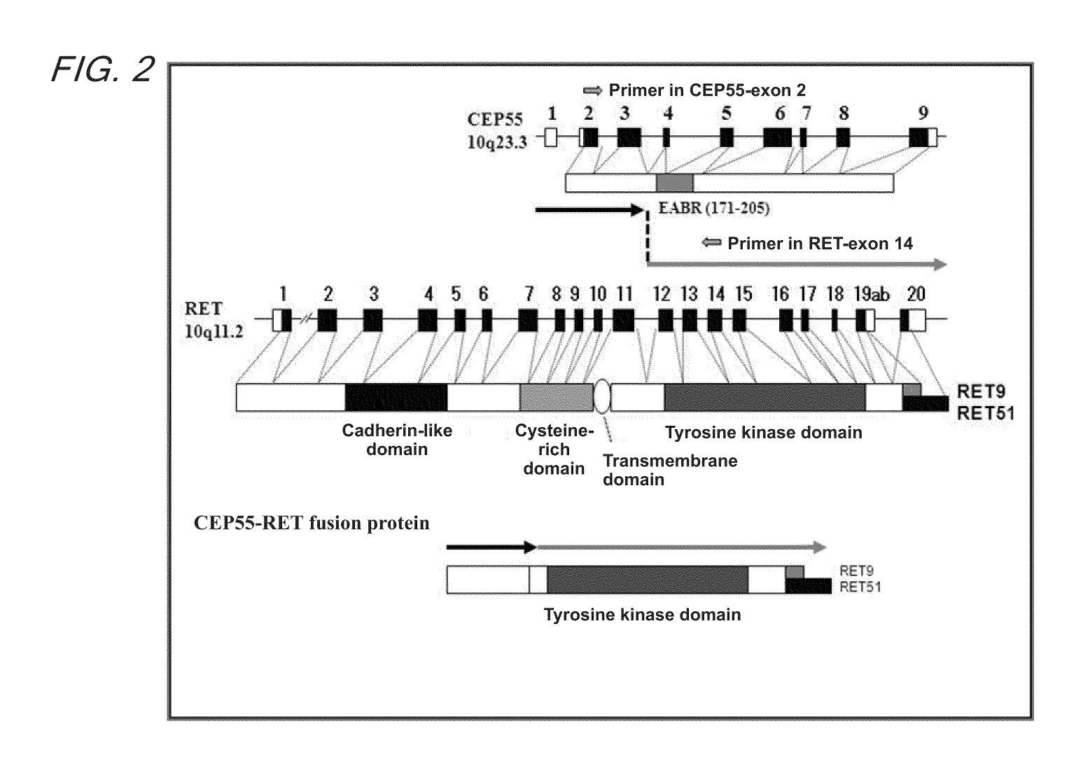

The “RET (rearranged during transfection) protein” according to the present invention is also referred to as “RET tyrosine kinase protein” or “RET receptor tyrosine kinase protein”, and means a protein encoded by the gene located at 10_{q}11.2 in humans. In this invention, the “RET protein”, as far as it is derived from humans, is typified by the protein consisting of the amino acid sequence of SEQ ID NO: 4 (RET51, RET isoform a) and the protein consisting of the amino acid sequence of SEQ ID NO: 6 (RET9, RET isoform c). The polynucleotide encoding the RET protein is typified by the polynucleotide consisting of the nucleotide sequence of SEQ ID NO: 3 (RET transcript variant 2) and the polynucleotide consisting of the nucleotide sequence of SEQ ID NO: 5 (RET transcript variant 4). As shown in FIG. 2, the protein consisting of the amino acid sequence of SEQ ID NO: 4 (RET51) and the protein consisting of the amino acid sequence of SEQ ID NO: 6 (RET9) share the common amino acid sequence starting with methionine at position 1 and ending with glycine at position 1063, but differ from each other in the sequence and length of the C-terminal moiety.

The CEP55-RET fusion polynucleotide is typified by a polynucleotide encoding a polypeptide in which the N-terminal moiety of CEP55 protein is fused with the C-terminal moiety of RET protein.

In the present invention, the “N-terminal moiety of CEP55 protein” is typified by a moiety comprising a region starting with methionine at position 1 and ending with glutamine at position 153 in the CEP55 protein. And the “C-terminal moiety of RET protein” is typified by a moiety comprising a tyrosine kinase domain in RET protein (refer to FIG. 2).

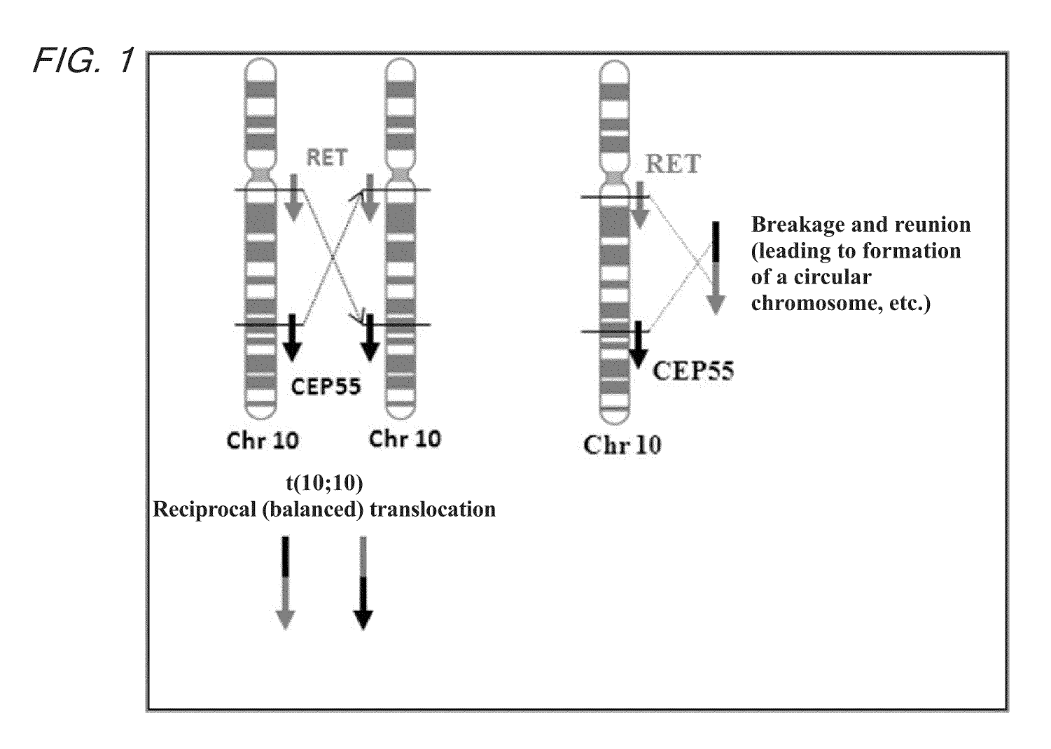

In the present invention, the “polynucleotide encoding a polypeptide in which the N-terminal moiety of CEP55 protein is fused with the C-terminal moiety of RET protein” is typically generated, as shown in FIGS. 1 to 4, by reciprocal translocation between two human chromosomes 10 or by breakage and reunion in human chromosome 10. More specifically, this term refers to a polynucleotide encoding a polypeptide in which a polypeptide region of the CEP55 protein that is encoded by exons 1-3 is fused with a polypeptide region of the RET protein that is encoded by exons 12-19b or 12-20 (e.g., the polypeptide consisting of the amino acid sequence of SEQ ID NO: 8 or 10). Said polynucleotide is exemplified by the polynucleotide consisting of the nucleotide sequence of SEQ ID NO: 7 or 9.

In the present invention, the amino acid sequences of “CEP55 protein” and “RET protein”, and the nucleotide sequences of the genes encoding said proteins can mutate in nature (i.e., in a non-artificial way). Thus, the amino acid sequence of the “CEP55-RET fusion polypeptide” and the nucleotide sequence of the “CEP55-RET fusion polynucleotide” can also mutate in nature (i.e., in a non-artificial way). Said amino acid sequences and nucleotide sequences may be artificially modified. Such mutants are also encompassed by this invention.

Certain exemplary mutants of the CEP55-RET fusion polypeptide include proteins consisting of an amino acid sequence derived from the amino acid sequence of SEQ ID NO: 8 or 10 by substitution, deletion, addition and/or insertion of one or more amino acids.

As used herein, the term “more” refers to generally 50 or fewer amino acids, preferably 30 or fewer amino acids, more preferably 10 or fewer amino acids, and particularly preferably several or fewer amino acids (for example, five or fewer amino acids, three or fewer amino acids, two or one amino acid, one amino acid).

Other exemplary mutants of the CEP55-RET fusion polypeptide include polypeptides encoded by a DNA that hybridizes under stringent conditions to a DNA consisting of the nucleotide sequence of SEQ ID NO: 7 or 9.

Exemplary high stringent hybridization conditions are 0.2×SSC at 65° C., and exemplary low stringent hybridization conditions are 2.0×SSC at 50° C.

Still other exemplary mutants of the CEP55-RET fusion polypeptide include polypeptides consisting of an amino acid sequence having at least 80% (for example, at least 85%, 90%, 95%, 97%, 99%) homology to the amino acid sequence of SEQ ID NO: 8.

Sequence homology can be determined using the BLASTX or BLASTP (amino acid level) program (Altschul, et al., J. Mol. Biol., 1990, 215: 403-410). This program is based on the algorithm BLAST developed by Karlin and Altschul (Proc. Natl. Acad. Sci. USA, 1990, 87: 2264-2268; and Proc. Natl. Acad. Sci. USA, 1993, 90: 5873-5877). When amino acid sequence analysis is performed using BLASTX, the parameters are typically set as follows: score=50 and wordlength=3. Amino acid sequence analysis using the Gapped BLAST program can be performed as per the descriptions in Altschul, et al. (Nucleic Acids Res., 1997, 25: 3389-3402). When amino acid sequence analysis is performed using the BLAST and Gapped BLAST programs, the default parameters of these programs are used. The specific procedures for conducting these analyses are known.

Exemplary mutants of the CEP55-RET fusion polynucleotide include polynucleotides encoding the above-mentioned mutants of the CEP55-RET fusion polypeptide, and polynucleotides encoding degenerate variants of said polypeptide which have no amino acid mutation.

Exemplary forms of the “CEP55-RET fusion polynucleotide” according to the present invention include mRNA, cDNA, and genomic DNA. It is possible for those skilled in the art using a known hybridization technique to isolate the “CEP55-RET fusion polynucleotide” from a cDNA library or genomic DNA library prepared from diffuse-type gastric cancer or other cancers that harbor a fusion gene between the CEP55 gene and the RET gene. The polynucleotide can also be prepared by amplification utilizing a known gene amplification technique (e.g., PCR), with the mRNA, cDNA or genomic DNA prepared from diffuse-type gastric cancer or other cancers being used as a template.

Furthermore, after the thus-prepared polynucleotide is inserted into an appropriate expression vector, the vector is introduced into a cell-free protein synthesis system (e.g., reticulocyte extract, wheat germ extract) and the system is incubated, or alternatively the vector is introduced into appropriate cells (e.g., E coli., yeast, insect cells, animal cells) and the resulting transformant is cultured; in either way, the CEP55-RET fusion polypeptide can be prepared.

As mentioned above, the “CEP55-RET fusion polypeptide” and “CEP55-RET fusion polynucleotide” according to the present invention encompasses, in a broad sense, both those having naturally occurring sequences (including those mutated in nature) and those having artificially modified sequences. However, it should be noted that the “CEP55-RET fusion polypeptide” and “CEP55-RET fusion polynucleotide”, particularly if these terms are used as an object of the detection as described below, mainly refer to those having naturally occurring sequences (including those mutated in nature).

<Method for Detecting the Presence or Absence of the CEP55-RET Fusion Polypeptide or the CEP55-RET Fusion Polynucleotide>

The present invention also provides a method for detecting the presence or absence of the CEP55-RET fusion polynucleotide or the CEP55-RET fusion polypeptide in a sample. The detection method of this invention comprises the steps of: (a) contacting the sample with an agent intended for specifically detecting the presence or absence of the polynucleotide or the polypeptide in the sample; and (b) detecting the presence or absence of the polynucleotide or the polypeptide.

For the purpose of the present invention, the term “sample” includes not only biological samples (for example, cells, tissues, organs, body fluids (e.g., blood, lymphs), digestive juices, sputum, bronchoalveolar/bronchial lavage fluids, urine, and feces), but also nucleic acid extracts from these biological samples (for example, genomic DNA extracts, mRNA extracts, and cDNA and cRNA preparations from mRNA extracts) and protein extracts. The sample may also be the one that is fixed with formalin or alcohol, frozen, or embedded in paraffin.

Further, the genomic DNA, mRNA, cDNA or protein can be prepared by those skilled in the art through considering various factors including the type and state of the sample and selecting a known technique suitable therefor.

In the present invention, “detecting the presence or absence of the CEP55-RET fusion polynucleotide or the CEP55-RET fusion polypeptide” can be performed on genomic DNAs encoding said fusion polypeptide, transcripts from said genomic DNAs, or translation products from said transcripts.

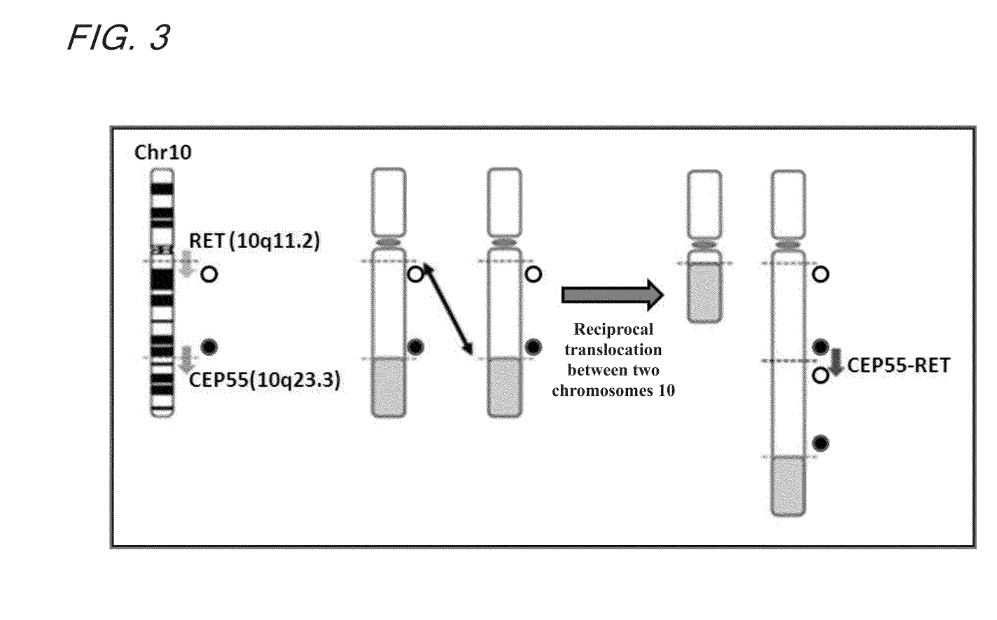

Since a genomic DNA encoding the CEP55-RET fusion polypeptide is formed by reciprocal translocation between two human chromosomes 10 or by breakage and reunion in human chromosome 10, “detecting the presence or absence of the CEP55-RET fusion polynucleotide” may be achieved by detecting this phenomenon of reciprocal translocation (refer to FIGS. 1 and 3). The detection of reciprocal translocation may be achieved, for example, by detecting a split of the portion consisting of the exon 3-coding region of the CEP55 gene and a region upstream from said coding region toward the 5′ end, from the portion consisting of the exon 4-coding region of the CEP55 gene and a region downstream from said coding region toward the 3′ end, or by detecting a split of the portion consisting of the exon 11-coding region of the RET gene and a region upstream from said coding region toward the 5′ end, from the portion consisting of the exon 12-coding region of the RET gene and a region downstream from said coding region toward the 3′ end.

“Detecting the presence of absence of the CEP55-RET fusion polynucleotide” according to the present invention can be performed using a known method. Exemplary known methods that can be used in the detection on the “genomic DNAs encoding said fusion polypeptide” include in situ hybridization (ISH) using fluorescence or other means, genomic PCR, direct sequencing, Southern blotting, and genome microarray analysis. Exemplary known methods that can be used in the detection on the “transcripts from said genomic DNAs” include RT-PCR, direct sequencing, Northern blotting, dot blotting, and cDNA microarray analysis.

According to in situ hybridization, genomic DNAs encoding the CEP55-RET fusion polypeptide can be detected by contacting a biological sample with the polynucleotide or polynucleotides noted below in (a) or (b), which have a chain length of at least 15 nucleotides:

(a) a polynucleotide or polynucleotides that are at least one probe selected from the group consisting of a probe that hybridizes to a polynucleotide encoding CEP55 protein and a probe that hybridizes to a polynucleotide encoding RET protein; or

(b) a polynucleotide that is a probe that hybridizes to a point of fusion between a polynucleotide encoding CEP55 protein and a polynucleotide encoding RET protein.

In relation to the detection of genomic DNAs encoding the CEP55-RET fusion polypeptide, the “polynucleotide encoding CEP55 protein” according to the present invention, as far as it is derived from humans, is typified by the gene consisting of the DNA sequence of positions 95256369 to 95288849 in the genome sequence identified by RefSeq ID: NC_{—}000010.10 (“CEP55 gene”).

The “polynucleotide encoding RET protein” according to the present invention, as far as it is derived from humans, is typified by the gene consisting of the DNA sequence of positions 43572517 to 43625799 in the genome sequence identified by RefSeq ID: NC_{—}000010.10 (“RET gene”).

However, the DNA sequences of the genes can vary in nature (i.e., in a non-artificial way) due to their mutations and the like. Thus, such naturally occurring mutants can also be encompassed by the present invention (the same applies hereinafter).

The polynucleotide(s) of (a) according to the present invention can be of any type as far as it is capable of detecting the presence of a genomic DNA encoding the CEP55-RET fusion polypeptide in the foregoing biological sample by hybridizing to a nucleotide sequence targeted by said polynucleotide, or more specifically to a polynucleotide encoding CEP55 protein or a polynucleotide encoding RET protein. The polynucleotide(s) of (a) is preferably any of the polynucleotides noted below in (a1) to (a3):

(a1) a combination of a polynucleotide that hybridizes to the portion consisting of the exon 3-coding region of the CEP55 gene and a region upstream from said coding region toward the 5′ end (this polynucleotide is to be hereinafter also referred to as “5′ CEP55 probe”), and a polynucleotide that hybridizes to the portion consisting of the exon 12-coding region of the RET gene and a region downstream from said coding region toward the 3′ end (this polynucleotide is to be hereinafter also referred to as “3′ RET probe”);

(a2) a combination of 5′ CEP55 probe and a polynucleotide that hybridizes to the portion consisting of the exon 4-coding region of the CEP55 gene and a region downstream from said coding region toward the 3′ end (this polynucleotide is to be hereinafter also referred to as “3′ CEP55 probe”); and

(a3) a combination of a polynucleotide that hybridizes to the portion consisting of the exon 11-coding region of the RET gene and a region upstream from said coding region toward the 5′ end (this polynucleotide is to be hereinafter also referred to as “5′ RET probe”), and 3′ RET probe.

In the present invention, the region to which the pair of polynucleotides of (a1) as used for in situ hybridization is to hybridize (such a region is to be hereinafter referred to as the “target nucleotide sequence”) is preferred to be a region extending for not more than 1000000 nucleotides from a point of fusion between a polynucleotide encoding CEP55 protein and a polynucleotide encoding RET protein, from the viewpoints of specificity for the target nucleotide sequence and detection sensitivity. And the region to which the pair of polynucleotides of (a2) or (a3) as used for in situ hybridization is to hybridize is preferred, from the same viewpoints, to be a region extending for not more than 1000000 nucleotides from a breakpoint in a polynucleotide encoding CEP55 protein or in a polynucleotide encoding RET protein.

In the present invention, the polynucleotide of (b) as used for in situ hybridization can be of any type as far as it is capable of detecting the presence of a genomic DNA encoding the CEP55-RET fusion polypeptide in the foregoing biological sample by hybridizing to a nucleotide sequence targeted by said polynucleotide, or more specifically to a point of fusion between a polynucleotide encoding CEP55 protein and a polynucleotide encoding RET protein. Typical examples of the polynucleotide of (b) are those which hybridize to a genomic DNA encoding a polynucleotide consisting of the nucleotide sequence of SEQ ID NO: 7 or 9, for example, those which hybridize to a point of fusion between a polynucleotide encoding CEP55 protein and a polynucleotide encoding RET protein.

Further, in the present invention, the polynucleotide or polynucleotides of (a) or (b) as used for in situ hybridization are preferred to be a group consisting of multiple types of polynucleotides which can cover the entire target nucleotide sequence, from the viewpoints of further improvement of specificity for the target nucleotide sequence and detection sensitivity. In such a case, the polynucleotides constituting said group each have a length of at least 15 nucleotides, preferably from 100 to 1000 nucleotides.

The polynucleotide or polynucleotides of (a) or (b) as used for in situ hybridization are preferably labeled with a fluorescent dye or other means for the purpose of detection. Examples of such a fluorescent dye include, but are not limited to, DEAC, FITC, R6G, TexRed, and Cy5. Aside from fluorescent dyes, the polynucleotide may also be labeled with a dye (chromogen) such as DAB or with silver or other means based on enzymatic metal deposition.

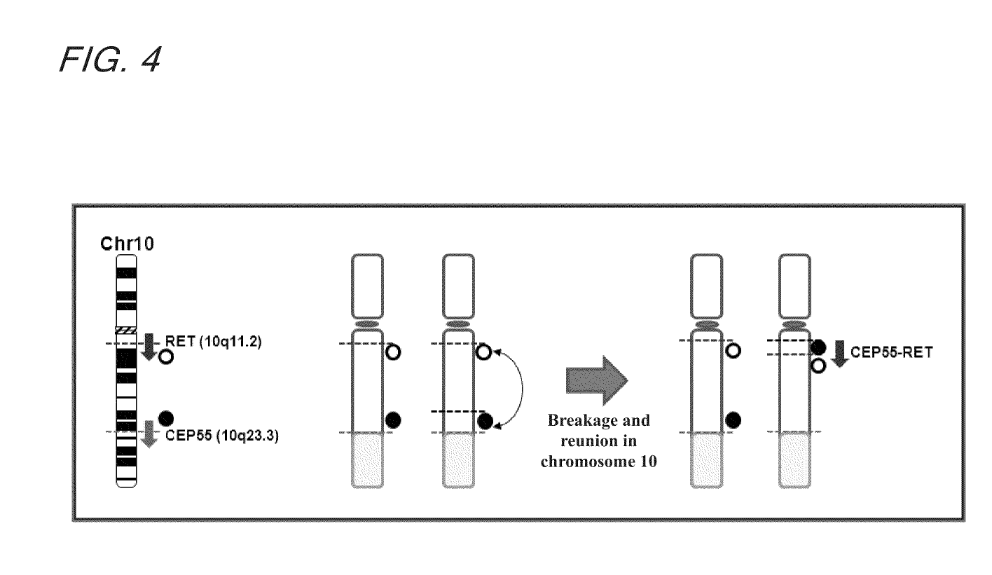

In the process of in situ hybridization, a probe specific for a polynucleotide encoding CEP55 protein and a probe specific for a polynucleotide encoding RET protein are preferably each labeled with a different dye. If in situ hybridization is carried out using the probe combination of (a1) which consists of probes labeled with different dyes, to thereby observe an overlap between signals emitted from the labels on these probes, then it can be determined that a genomic DNA encoding the CEP55-RET fusion polypeptide has been detected successfully (refer to FIGS. 3 and 4). Also, if in situ hybridization is carried out using the probe combination of (a2) or (a3) which consists of probes labeled with different dyes, to thereby observe a split between signals emitted from the labels on these probes, then it can be determined that a genomic DNA encoding the CEP55-RET fusion polypeptide has been detected successfully.

Polynucleotide labeling can be effected by a known method. For example, the polynucleotides can be labeled by nick translation or random priming, in which the polynucleotides are caused to incorporate substrate nucleotides labeled with a fluorescent dye or other means.

The conditions for contacting the foregoing biological sample with the polynucleotide(s) of (a) or (b) in the process of in situ hybridization can vary with various factors including the length of said polynucleotide(s); and exemplary high stringent hybridization conditions are 0.2×SSC at 65° C., and exemplary low stringent hybridization conditions are 2.0×SSC at 50° C. Those skilled in the art could realize comparable stringent hybridization conditions to those mentioned above, by appropriately selecting salt concentration (e.g., SSC dilution rate), temperature, and various other conditions including concentrations of surfactant (e.g., NP-40) and formamide, and pH.

Aside from in situ hybridization, other examples of the method for detecting a genomic DNA encoding the CEP55-RET fusion polypeptide using the polynucleotide(s) of (a) or (b) include Southern blotting, Northern blotting and dot blotting. According to these methods, the fusion gene is detected by hybridizing the polynucleotide(s) of (a) or (b) to a membrane in which a nucleic acid extract from the foregoing biological sample has been transcribed. In the case of using the polynucleotide(s) of (a), if a polynucleotide that hybridizes to a polynucleotide encoding CEP55 protein and a polynucleotide that hybridizes to a polynucleotide encoding RET protein recognize the same band present in the membrane, then it can be determined that a genomic DNA encoding the CEP55-RET fusion polypeptide has been detected successfully.

Additional examples of the method for detecting a genomic DNA encoding the CEP55-RET fusion polypeptide using the polynucleotide of (b) include genome microarray analysis and DNA microarray analysis. According to these methods, the genomic DNA is detected by preparing an array in which the polynucleotide of (b) is immobilized on a substrate and bringing the foregoing biological sample into contact with the polynucleotide immobilized on the array.

In the process of PCR or sequencing, the polynucleotides noted below in (c) can be used to specifically amplify part or all of the CEP55-RET fusion polynucleotide using a DNA (genomic DNA, cDNA) or RNA prepared from the foregoing biological sample as a template:

(c) polynucleotides that are a pair of primers designed to sandwich a point of fusion between a polynucleotide encoding CEP55 protein and a polynucleotide encoding RET protein.

The “polynucleotides that are a pair of primers” refers to a primer set designed such that in the foregoing fusion polynucleotide or the like to be targeted, one of the primers hybridizes to a region of the CEP55 gene and the other primer hybridizes to a region of the RET gene. These polynucleotides have a length of generally 15-100 nucleotides, preferably 17-30 nucleotides.

Also, it is preferred from the viewpoints of the accuracy and sensitivity of PCR detection that the polynucleotides of (c) according to the present invention should each consist of a sequence complementary to the nucleotide sequence of said fusion polynucleotide which extends for not more than 5000 nucleotides from a point of fusion between a polynucleotide encoding CEP55 protein and a polynucleotide encoding RET protein.

The “polynucleotides that are a pair of primers” can be designed by a known method as appropriate based on the nucleotide sequence of the CEP55-RET fusion polynucleotide or the like to be targeted. Exemplary known methods include a method using the Primer Express® software (ABI).

Preferred examples of the “polynucleotides that are a pair of primers” are preferably the polynucleotides noted below in (c1):

(c1) a combination of a polynucleotide that hybridizes to the portion consisting of the exon 3-coding region of the CEP55 gene and a region upstream from said coding region toward the 5′ end (this polynucleotide is to be hereinafter also referred to as “5′ CEP55 primer”), and a polynucleotide that hybridizes to the portion consisting of the exon 12-coding region of the RET gene and a region downstream from said coding region toward the 3′ end (this polynucleotide is to be hereinafter also referred to as “3′ RET primer”).

For example, in the process of detection of the CEP55-RET fusion polynucleotide, primers are each designed for exon 2 containing the start codon of the CEP55 gene or for exon 14 in the kinase region of the RET gene, so that detection can be made of all variants containing the tyrosine kinase region of RET protein, and all fusion genes with the CEP55 gene (refer to FIG. 2).

In the present invention, the method for detecting a translation product of the CEP55-RET fusion polynucleotide can be exemplified by immunostaining, Western blotting, ELISA, flow cytometry, immunoprecipitation, and antibody array analysis. These methods use an antibody binding to the CEP55-RET fusion polypeptide. Examples of such an antibody include an antibody specific to a polypeptide containing a point of fusion between CEP55 protein and RET protein (hereinafter also referred to as the “fusion point-specific antibody”), an antibody binding to a polypeptide consisting of a region of CEP55 protein that extends toward the N-terminus with respect to said point of fusion (hereinafter also referred to as the “CEP55-N terminal antibody”), and an antibody binding to a polypeptide consisting of a region of RET protein that extends toward the C-terminus with respect to said point of fusion (hereinafter also referred to as the “RET protein-C terminal antibody”). As referred to herein, the “fusion point-specific antibody” means an antibody that specifically binds to a polypeptide containing said point of fusion but does not bind to either wild-type (normal) CEP55 protein or wild-type (normal) RET protein.

The CEP55-RET fusion polypeptide can be detected by the fusion point-specific antibody or a combination of the CEP55-N terminal antibody and the RET protein-C terminal antibody.

The “antibody binding to the CEP55-RET fusion polypeptide” can be prepared by those skilled in the art through selection of a known method as appropriate. Examples of such a known method include: a method in which the polypeptide comprising the C-terminal moiety of RET protein, the CEP55-RET fusion polypeptide, the polypeptide comprising the N-terminal moiety of CEP55 protein, and/or the like are inoculated into an immune animal, the immune system of the animal is activated, and then the serum (polyclonal antibody) of the animal is collected; as well as monoclonal antibody preparation methods such as hybridoma method, recombinant DNA method, and phage display method. If an antibody having a labeling agent attached thereto is used, a target protein can be directly detected by detecting this label. The labeling agent is not particularly limited as long as it is capable of binding to an antibody and is detectable, and examples include peroxidase, β-D-galactosidase, microperoxidase, horseradish peroxidase (HRP), fluorescein isothiocyanate (FITC), rhodamine isothiocyanate (RITC), alkaline phosphatase, biotin, and radioactive materials. Aside from the direct detection of a target protein using an antibody having a labeling agent attached thereto, the target protein can also be indirectly detected using a secondary antibody having a labeling agent attached thereto, Protein G or A, or the like.

<Method for Determining the Effectiveness of Cancer Treatments with a RET Tyrosine Kinase Inhibitor>

As disclosed below in Examples, gene fusions between the CEP55 gene and the RET gene are believed to induce activation of RET protein and contribute to malignant transformation of cancers and other pathological conditions. Thus, it is highly probable that cancer patients with detection of such a fusion are responsive to treatments with a RET tyrosine kinase inhibitor.

Therefore, the present invention provides a method for determining the effectiveness of a cancer treatment with a RET tyrosine kinase inhibitor, the method comprising the step of detecting the presence or absence of the CEP55-RET fusion polynucleotide or the CEP55-RET fusion polypeptide in a sample isolated from a patient, wherein in a case where the presence of the polynucleotide or polypeptide is detected, the cancer treatment with the RET tyrosine kinase inhibitor is determined to be highly effective in the patient.

For the purpose of the present invention, the “patient” can be not only a human suffering from a cancer but also a human suspected of having a cancer. The “cancer” to which the method of this invention is to be applied is not particularly limited as long as it is a cancer with expression of the CEP55-RET fusion gene. The cancer is preferably diffuse-type gastric cancer. Collection of a biological sample from a patient can be performed by a known method depending on the type of the biological sample.

For the purpose of the present invention, the “RET tyrosine kinase inhibitor”, the cancer treatment with which is to be evaluated for effectiveness, is not particularly limited as long as it is a substance capable of directly or indirectly suppressing the function of RET protein. Examples of known RET tyrosine kinase inhibitors that can be applied to the present invention include: 4-(4-bromo-2-fluoroanilino)-6-methoxy-7-(1-methylpiperidin-4-ylmethoxy)quinazoline (generic name: vandetanib; a compound targeting VEGFR, EGFR and RET); 4-[4-[3-[4-Chloro-3-(trifluoromethyl)phenyl]ureido]phenoxy]-N-methylpyridine-2-carboxamide (generic name: sorafenib; a compound targeting BRAF, RET, etc.); N-[2-(Diethylamino)ethyl]-5-[(Z)-(5-fluoro-2-oxo-1,2-dihydro-3H-indol-3-ylidene)methyl]-2,4-dimethyl-1H-pyrrole-3-carboxamide mono[(2S)-2-hydroxysuccinate] (generic name: sunitinib; a compound targeting PDGFR, VEGFR, RET, etc.); N-(3,3-Dimethylindolin-6-yl)-2-(pyridin-4-ylmethylamino)nicotinamide (generic name: motesanib; a compound targeting PDGFR, VEGFR, RET, etc.); and N-(4-(6,7-dimethoxyquinolin-4-yloxy)phenyl)-N-(4-fluorophenyl)cyclopropane-1,1-dicarboxamide (generic name: XL184/cabozantinib; a compound targeting MET, RET, etc.).

The definition of the term “sample”, the method for extracting a DNA, an RNA or the like from the sample, the procedure for detecting the presence or absence of the CEP55-RET fusion polynucleotide or the CEP55-RET fusion polypeptide, and other related information are as described above.

If the presence of the CEP55-RET fusion polynucleotide or the CEP55-RET fusion polypeptide in a sample isolated from a patient is detected according to the inventive method, the patient will be determined to be highly responsive to a cancer treatment with a RET tyrosine kinase inhibitor. If the presence of the polynucleotide or polypeptide is not detected, the patient will be determined to be less responsive to a cancer treatment with a RET tyrosine kinase inhibitor.

<Agents for Detecting the Presence or Absence of the CEP55-RET Fusion Polynucleotide or the CEP55-RET Fusion Polypeptide, and Agents for Determining the Effectiveness of Cancer Treatments with a RET Tyrosine Kinase Inhibitor>

As described above, the polynucleotides noted below in (a) to (c), which each have a chain length of at least 15 nucleotides, can be used advantageously for detecting the presence or absence of the CEP55-RET fusion polynucleotide. Thus, said polynucleotides can also be used advantageously for determining the effectiveness of cancer treatments with a RET tyrosine kinase inhibitor.

(a) A polynucleotide or polynucleotides that are at least one probe selected from the group consisting of a probe that hybridizes to a polynucleotide encoding CEP55 protein and a probe that hybridizes to a polynucleotide encoding RET protein;

(b) a polynucleotide that is a probe that hybridizes to a point of fusion between a polynucleotide encoding CEP55 protein and a polynucleotide encoding RET protein; and

(c) polynucleotides that are a pair of primers designed to sandwich a point of fusion between a polynucleotide encoding CEP55 protein and a polynucleotide encoding RET protein.

Said polynucleotides each have a nucleotide sequence complementary to a particular nucleotide sequence of a target gene. As referred to herein, the term “complementary” may not necessarily refer to perfect complementarity as long as hybridization is achieved. Said polynucleotides have generally at least 80% homology, preferably at least 90% homology, more preferably at least 95% homology, and particularly preferably 100% homology with such a particular nucleotide sequence.

The polynucleotides of (a) to (c) may be a DNA or a RNA, or may be such that part or all of the nucleotides are substituted by an artificial nucleic acid such as PNA (polyamide nucleic acid: a peptide nucleic acid), LNA™ (Locked Nucleic Acid; a bridged nucleic acid), ENA® (2′-O,4′-C-Ethylene-bridged Nucleic Acid), GNA (glycerol nucleic acid) or TNA (threose nucleic acid).

As described above, the antibody binding to an CEP55-RET fusion polypeptide can be used advantageously for detecting translation products (CEP55-RET fusion polypeptides) of the CEP55-RET fusion polypeptide.

The agents of the present invention can contain not only the foregoing substance (e.g., polynucleotide, antibody) as an active ingredient but also other pharmacologically acceptable components. Such other components include buffer agents, emulsifying agents, suspending agents, stabilizing agents, antiseptic agents, and physiological saline. As buffer agents, there can be used phosphates, citrates, acetates and the like. As emulsifying agents, there can be used gum arabic, sodium alginate, tragacanth, and the like. As suspending agents, there can be used glyceryl monostearate, aluminum monostearate, methylcellulose, carboxymethyl cellulose, hydroxymethyl cellulose, sodium lauryl sulfate, and the like. As stabilizing agents, there can be used propylene glycol, diethylene sulfite, ascorbic acid, and the like. As antiseptic agents, there can be used sodium azide, benzalkonium chloride, paraoxybenzoic acid, chlorobutanol, and the like.

A specimen containing the inventive polynucleotide or antibody may also be combined with other specimens such as a substrate required for detecting a label attached to the polynucleotide or the antibody, a positive control (e.g., CEP55-RET fusion polynucleotide, CEP55-RET fusion polypeptide, or cells bearing the same), a negative control, a counterstaining reagent for use for in situ hybridization or the like (e.g., DAPI), a molecule required for antibody detection (e.g., secondary antibody, Protein G, Protein A), and a buffer solution for use in sample dilution or washing; by combining such specimens, a kit for use in the method of the present invention can be provided. The inventive kit can contain instructions for use thereof. Therefore, the present invention also provides the foregoing kit for use in the inventive method.

<Method for Treatment of Cancer, and Therapeutic Agents for Cancer>

As described above, if the presence of the CEP55-RET fusion polynucleotide or the CEP55-RET fusion polypeptide is detected in a patient by the method of the present invention, the patient is considered to be highly responsive to a cancer treatment with a RET tyrosine kinase inhibitor. Thus, efficient cancer treatment is possible by administering a RET tyrosine kinase inhibitor selectively to those cancer patients who carry the CEP55-RET fusion gene. Therefore, this invention provides a method for treatment of cancer, comprising the step of administering a RET tyrosine kinase inhibitor to a patient in whom a cancer treatment with the RET tyrosine kinase inhibitor has been determined to be highly effective according to the foregoing determination method of this invention.

Further, the present invention provides a therapeutic agent for cancer, comprising a RET tyrosine kinase inhibitor as an active ingredient, wherein the therapeutic agent is to be administered to a patient in whom a cancer treatment with the RET tyrosine kinase inhibitor has been determined to be highly effective according to the foregoing determination method of this invention.

As described above, the “RET tyrosine kinase inhibitor” is not particularly limited as long as it is a substance capable of directly or indirectly suppressing the function of RET protein. Examples of known RET tyrosine kinase inhibitors that can be applied to the present invention are as given above.

The dosage form for administering a RET tyrosine kinase inhibitor to a patient is selected as appropriate depending on various factors including the type of the inhibitor and the type of cancer, and examples of the dosage form that can be adopted include oral, intravenous, intraperitoneal, transdermal, intramuscular, intratracheal (aerosol), rectal, intravaginal and other administrations.

### EXAMPLES

Hereunder, the present invention will be more specifically described on the basis of Examples, but this invention is not limited to the examples given below.

<Test Samples>

Thirteen surgically resected and frozen specimens of diffuse-type gastric cancer (DGC) were used as an object to be tested. As a negative control, normal gastric mucosal tissues were also used.

<RNA Extraction>

The tumor tissues cryopreserved in liquid nitrogen were pulverized with cryoPREP (product name: CP02; Covaris). Then, total RNA was extracted from the pulverized tumor tissues using a total RNA purification kit (product name: RNAeasy; QIAGEN).

<RNA Sequencing>

Library synthesis was performed with an mRNAseq sample preparation kit (Illumina) using 2 μg of the total RNA prepared hereinabove. More specifically, 2 μg of the total RNA was fragmented by treatment at 94° C. for 5 minutes and subjected to cDNA synthesis. Next, a sequencing adapter was ligated to each end of the resulting cDNA, which was then subjected to electrophoresis on agarose gel, followed by purification. PCR was performed using the purified cDNA as a template to construct a 300 bp cDNA library. The sequences of 50 bp from both ends of the constructed cDNA library were sequenced using a high-throughput sequencer (GA2X; Illumina).

<Analysis of Sequence Information>

The obtained sequence information, from which overlapping clones generated by PCR were excluded, was mapped to known databases (Refseq and Ensemble) using the Bowtie software to extract clones whose end sequences are derived from different genes (refer to Non-patent Document 4).

<Verification by RT-PCR and Sanger Method>

The same total RNA as used in RNA sequencing was subjected to cDNA synthesis anew using a reverse transcriptase (SuperScript III First-Strand Synthesis System; Invitrogen). PCR primers were synthesized based on the resulting sequences. PCR was performed using ExTaq HS (Takara), and then the PCR products were verified by electrophoresis. Further, the PCR products were extracted from the agarose gel and sequenced by the Sanger method using the same primers, the BigDye Terminator v3.1 Cycle Sequencing Kit (Applied Biosystems) and the ABI 3730 Sequencer. The obtained results were used to identify points of fusion of fusion genes and reading frames.

The conditions for reverse transcription reaction and PCR are as follows.

<Reverse Transcription Reaction>

First, 5 μg (8 μL) of the total RNA was mixed with 1 μL of Random Hexamer Primer (50 ng/μL) and 1 μL of 10 mM dNTP Mix, and the mixture was reacted at 65° C. for 5 minutes and quenched on ice. Next, 2 μL of 10×RT buffer, 4 μL of 25 mM MgCl₂, 2 μL of 0.1 M DTT, 1 μL of RNaseOUT (40 U/μL), and 1 μL of SuperScript III RT (200 U/μL) were added in this order, and reverse transcription reaction was effected under the following conditions: 25° C. for 10 minutes, 50° C. for 50 minutes, 85° C. for 5 minutes, and 4° C. for 5 minutes. To the resulting reverse transcripts, 1 μL of RNaseH was added to digest the total RNA at 37° C. over 20 minutes. The completed reverse transcription reactions were stored at −20° C. until use.

<PCR>

First, 2 μL of the hereinabove prepared 1st strand cDNA, 1 μL of 10×ExTaq buffer, 1.2 μL of 2.5 mM dNTPs, 4.7 μL of H₂O, 0.1 μL of ExTaq HS, and 1 μL of 2 μM CF/CR primer pair were mixed, and the mixture was subjected to PCR under the following conditions: the reaction started with 95° C. for 3 minutes, followed by 35 cycles consisting of 94° C. for 30 seconds, 58° C. for 30 seconds, and 72° C. for 30 seconds, and ended with 72° C. for 5 minutes.

### Example 1

### Identification of Novel Kinase Fusion Genes by RNA Sequencing

From each of 13 clinical DGC specimens, there were obtained at least 8.0×10⁷ paired nucleotide sequences, with overlapping clones generated by PCR being excluded. As the result of comparison of these sequences to existing gene databases, the following two candidates for fusion gene between the CEP55 gene and the RET gene, as shown in FIG. 2, were detected each in one specimen: a polynucleotide encoding a polypeptide in which CEP55 protein and RET9 protein are fused together, and a polynucleotide encoding a polypeptide in which CEP55 protein and RET51 protein are fused together (hereinafter also referred to the “CEP55-RET fusion gene”).

[Verification by RT-PCR and Sanger Sequencing]

Next, the same specimens were verified for the presence of the fusion gene of interest by RT-PCR and Sanger sequencing. As a result, RT-PCR revealed that specimen-specific amplification of said fusion gene was observed, or in other words that said fusion gene was expressed only in DGC tissues and not in normal gastric mucosal tissues.

Further, sequencing of the obtained PCR products revealed that, as shown in FIG. 2 and SEQ ID NOs: 7 and 9, in the CEP55-RET fusion gene, exon 3 in the CEP55 gene (i.e., the polynucleotide consisting of the nucleotide sequence of positions 488 to 763 in SEQ ID NO: 1, 7 or 9) and exon 12 in the RET gene (i.e., the polynucleotide consisting of the nucleotide sequence of positions 2327 to 2474 in SEQ ID NO: 3 or 5) are directly bound together, whereby the two genes are fused together without inconsistency in their reading frames.

Accordingly, the results presented hereinabove suggested that since the CEP55 and RET genes are present in the same chromosome 10 in the same orientation, these genes are fused by reciprocal translocation between two chromosomes 10 (refer to the left half of FIG. 1) or by breakage and reunion in chromosome 10 (refer to the right half of FIG. 1) in a specific manner to cancer cells such as DGC. In fact, analysis of the nucleotide sequences of the fusion sites in genomic DNAs obtained from cancer tissues from undifferentiated gastric cancer patients demonstrated that in the cancer tissues from said patients, the CEP55 and RET genes are fused together by breakage and reunion in chromosome 10.

### Example 2

### Analyses of the Function of the CEP55-RET Fusion Polypeptides, and of the Effectiveness of RET Tyrosine Kinase Inhibitors Against Cancer Cells Expressing Said Polypeptides

It is considered that the above-mentioned gene fusion induces activation of RET protein, and that this activation induces activation of a downstream signal, thereby causing canceration of cells. Therefore, it is conceivable that RET tyrosine kinase inhibitors may be therapeutically effective in patients with such activations. In order to verify these points, analysis of the CEP55-RET fusion gene was made by following appropriate conventional methods, as described below.

First, a cDNA encoding a FLAG epitope tag was joined to the 5′ end of the cDNA of the CEP55-RET fusion gene (a polynucleotide encoding a polypeptide in which CEP55 and RET51 are fused together) obtained from undifferentiated gastric cancer patients' cancer tissues, by aligning their translation reading frames with each other. The resulting product was cloned into the pMXs retroviral vector.

Also, site-directed mutagenesis was performed on this vector to construct a vector encoding a kinase activity mutant with substitution of one amino acid in a RET kinase region (KD-mutated CEP55-RET fusion polypeptide: K758M).

Next, normal murine fibroblast line NIH-3T3 cells were infected with each of the thus-prepared retroviral vectors to obtain cell lines stably expressing the wild-type or the mutated CEP55-RET fusion polypeptide.

Furthermore, a retroviral vector encoding a RET fusion gene detected in lung adenocarcinoma (fusion gene between the KIF5B gene and the RET gene; hereinafter also referred to as the “KIF5B-RET fusion gene”), as well as a retroviral vector encoding a kinase activity mutant with substitution of lysine for methionine in a RET kinase region, were constructed by the same procedures as described above. NIH-3T3 cells were infected with each of these retroviral vectors to prepare cell lines stably expressing the wild-type or the mutated KIF5B-RET fusion polypeptide. These cell lines were subjected to the tests described below as a control group.

As for the KIF5B-RET fusion gene, reference should be made to Kohno T., et al., Nature Medicine, published online on Feb. 12, 2012, vol. 18, no. 3, p. 375-377. The “KIF5B-RET fusion variant 1” as referred to therein is the KIF5B-RET fusion gene which was used as a control in this working example.

In this specification, the CEP55-RET fusion gene and the KIF5B-RET fusion gene (or the CEP55-RET fusion polypeptide and the KIF5B-RET fusion polypeptide) are also collectively referred to as the “RET fusion genes (or RET fusion polypeptides)”.

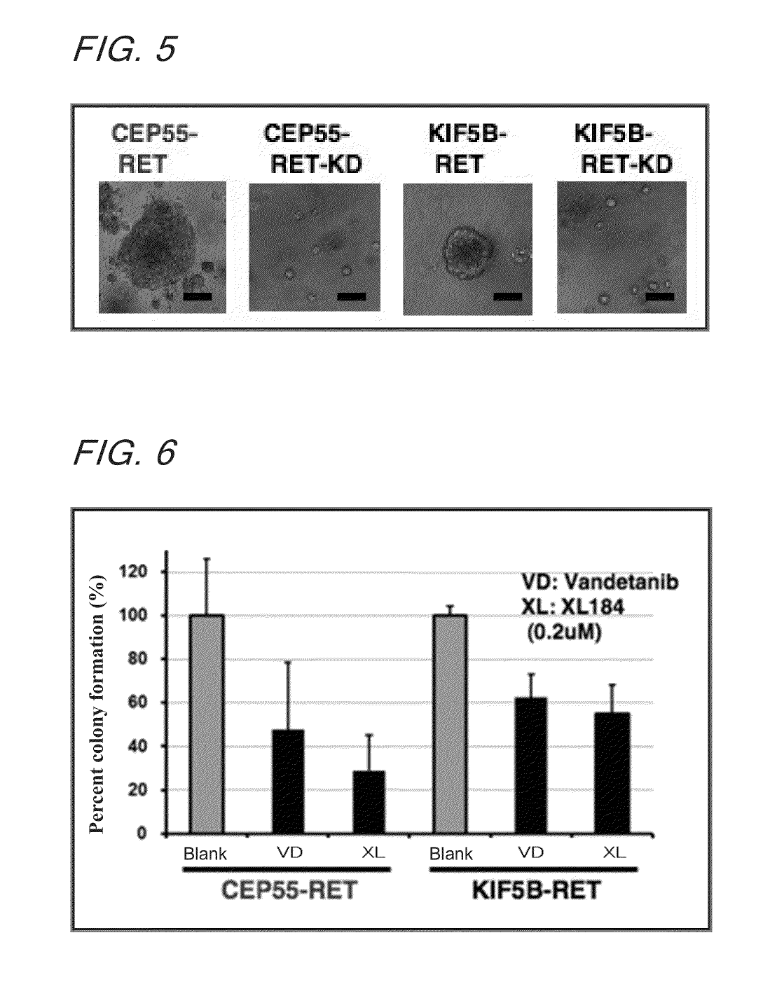

The cell lines stably expressing the wild-type or the mutated RET fusion polypeptide were each seeded in a soft agar medium (at an agar concentration in medium of 4 mg/mL; the same applies hereunder) and evaluated for their anchorage-independent colony-forming ability to thereby investigate the transforming ability of the RET fusion polypeptides. The results are shown in FIG. 5.

Further, the cell lines stably expressing the wild-type RET fusion polypeptides were each seeded in a soft agar medium supplemented with a low-molecular-weight RET tyrosine kinase inhibitor (vandetanib or XL184) and evaluated for their anchorage-independent colony-forming ability to thereby investigate the effect of the RET tyrosine kinase inhibitors against transformation induced by the RET fusion polypeptides. The results are shown in FIG. 6.

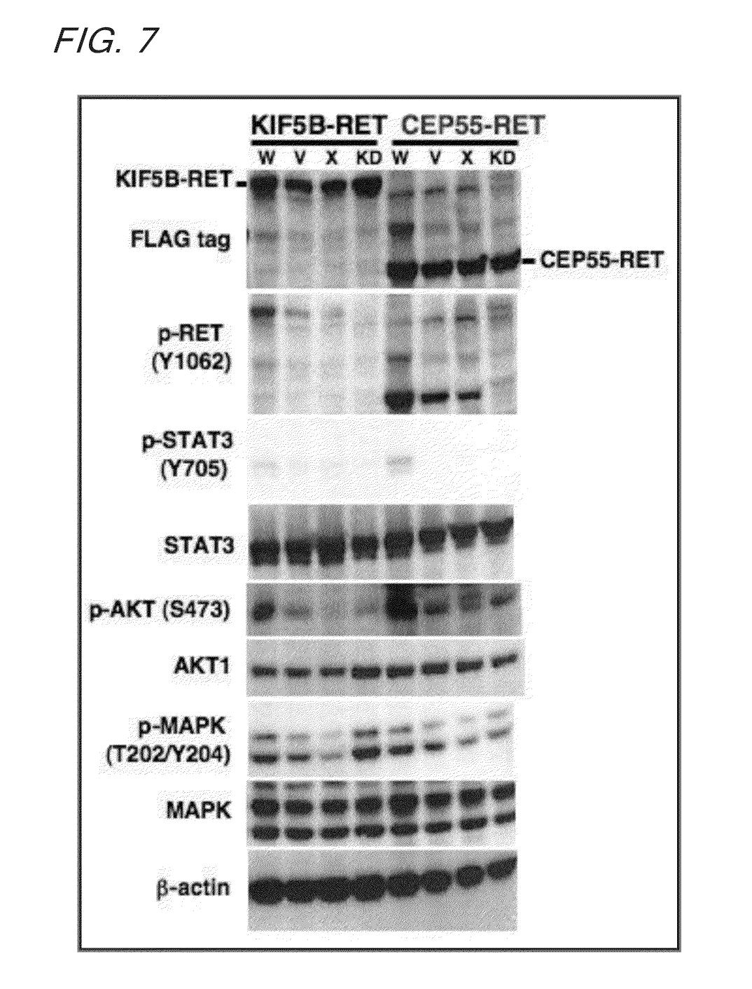

In addition, the cell lines stably expressing the wild-type or the mutated RET fusion polypeptide were each cultured in a liquid medium, subjected to serum starvation, and then cultured again with the medium being replaced with the one supplemented with a RET tyrosine kinase inhibitor (vandetanib or XL184). Protein from each of the thus-obtained cell lines was extracted and subjected to Western blotting analysis using antibodies against various phosphorylated or unphosphorylated proteins to thereby analyze the downstream signals of the RET fusion polypeptides. The results are shown in FIG. 7.

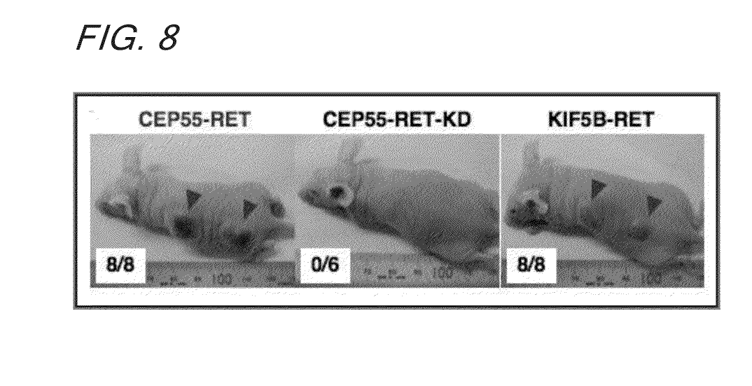

Furthermore, the cell lines stably expressing the wild-type or the mutated CEP55-RET fusion polypeptide, and the cell line stably expressing the wild-type KIF5B-RET fusion polypeptide, were each subcutaneously transplanted into immunodeficient mice (BALB/c-nu/nu) at a dose of 1×10⁶ cells per spot to thereby investigate the in vivo tumorigenic ability of the cells expressing these RET fusion polypeptides. The results are shown in FIG. 8.

As is evident from the results shown in FIG. 5, NIH-3T3 cells showed anchorage-independent colony formation due to the expression of the CEP55-RET fusion polypeptide. On the other hand, it was found that the anchorage-independent colony-forming ability of the CEP55-RET fusion polypeptides is significantly suppressed by inactivating the kinase activity of RET protein.

It was also found that, as shown in FIG. 6, the anchorage-independent colony-forming ability of the CEP55-RET fusion polypeptides is significantly suppressed by using a RET tyrosine kinase inhibitor.

As is evident from the results shown in FIG. 7, NIH-3T3 cells showed strong phosphorylation of the RET kinase domain due to the expression of the CEP55-RET fusion polypeptide (refer to the lanes labeled as “W” in the “p-RET (Y1062)” row of FIG. 7). It was also found that this phosphorylation is significantly suppressed by treatment with a RET tyrosine kinase inhibitor or by inactivation of the kinase activity of RET protein (refer to the lanes labeled as “V” and “X”, or “KD” in the “p-RET (Y1062)” row of FIG. 7).

As shown in FIG. 7, the phosphorylation of AKT was strongly induced by the CEP55-RET fusion polypeptide (refer to the lanes labeled as “W” in the “p-AKT (S473)” row of FIG. 7). It was also found that phosphorylation of STATS and MAPK is increased by said fusion peptide (refer to the lanes labeled as “W” in the “p-STATS (Y705)” and “p-MAPK (T202/Y204705)” rows of FIG. 7), but that the phosphorylation of these factors is also significantly suppressed by treatment with a RET tyrosine kinase inhibitor or by inactivation of the kinase activity of RET protein (refer to the lanes labeled as “V” and “X”, or “KD” in the “p-RET (Y1062)”, “p-STAT3 (Y705)” and “p-MAPK (T202/Y204705)” rows of FIG. 7).

As shown in FIG. 8, tumorigenesis was observed within 14 days after NIH-3T3 cells expressing the wild-type CEP55-RET fusion polypeptide were subcutaneously transplanted into immunodeficient mice. On the other hand, NIH-3T3 cells expressing the mutated CEP55-RET fusion polypeptide were also subcutaneously transplanted into immunodeficient mice, but no tumorigenesis was observed at all for 30 days after the transplantation.

Accordingly, these findings demonstrated that the CEP55-RET fusion gene serves as an oncogene with transforming ability, and that this transformation requires the activation of the RET kinase activity.

It is also found that the transforming ability of the CEP55-RET fusion polypeptide is suppressed using a RET tyrosine kinase inhibitor by inhibiting the activations of the RET tyrosine kinase in said fusion polypeptide and its downstream signals such as AKT.

### INDUSTRIAL APPLICABILITY

As described above, the present invention enables detection of polynucleotides encoding fusion polypeptides between CEP55 protein or part thereof and RET protein or part thereof, as well as expression products of said polynucleotides, and also this detection makes it possible to predict the effectiveness of cancer treatments with a RET tyrosine kinase inhibitor. This fusion induces activation of RET protein, thereby causing canceration of cells. This fusion induces the activation of RET protein, and in turn causes canceration of cells. Further, as demonstrated above, said RET activation and canceration can be significantly suppressed by using a RET tyrosine kinase inhibitor. Therefore, since the fusion between the CEP55 gene and the RET gene can be targeted by RET tyrosine kinase inhibitors, the present invention is very useful in improving the efficiency of cancer treatments.

1. A polynucleotide encoding a polypeptide in which CEP55 protein or part thereof and RET protein or part thereof are fused together.

2. A polypeptide encoded by the polynucleotide according to claim 1.

3. A method for detecting the presence or absence in a sample of the polynucleotide according to claim 1, the method comprising the steps of:
(a) contacting the sample with an agent intended for specifically detecting the presence or absence of the polynucleotide in the sample; and
(b) detecting the presence or absence of the polynucleotide.

4. An agent for detecting the presence or absence in a sample of a polynucleotide encoding a polypeptide in which CEP55 protein or part thereof and RET protein or part thereof are fused together for use in the method according to claim 3, the agent comprising a polynucleotide or polynucleotides as set forth below in any one of (a) to (c), the polynucleotide or polynucleotides having a chain length of at least 15 nucleotides:
(a) a polynucleotide or polynucleotides that are at least one probe selected from the group consisting of a probe that hybridizes to a polynucleotide encoding CEP55 protein and a probe that hybridizes to a polynucleotide encoding RET protein;
(b) a polynucleotide that is a probe that hybridizes to a point of fusion between a polynucleotide encoding CEP55 protein and a polynucleotide encoding RET protein; and
(c) polynucleotides that are a pair of primers designed to sandwich a point of fusion between a polynucleotide encoding CEP55 protein and a polynucleotide encoding RET protein.

5. A method for determining the effectiveness of a cancer treatment with a RET tyrosine kinase inhibitor, the method comprising the step of detecting the presence or absence in a sample isolated from a patient of the polynucleotide according to claim 1, wherein in a case where the presence of the polynucleotide is detected, the cancer treatment with the RET inhibitor is determined to be highly effective in the patient.

6. An agent for determining the effectiveness of a cancer treatment with a RET inhibitor by the method according to claim 5, the agent comprising a polynucleotide or polynucleotides as set forth below in any one of (a) to (c), the polynucleotide or polynucleotides having a chain length of at least 15 nucleotides:
(a) a polynucleotide or polynucleotides that are at least one probe selected from the group consisting of a probe that hybridizes to a polynucleotide encoding CEP55 protein and a probe that hybridizes to a polynucleotide encoding RET protein;
(b) a polynucleotide that is a probe that hybridizes to a point of fusion between a polynucleotide encoding CEP55 protein and a polynucleotide encoding RET protein; and
(c) polynucleotides that are a pair of primers designed to sandwich a point of fusion between a polynucleotide encoding CEP55 protein and a polynucleotide encoding RET protein.

7. A method for treatment of cancer, comprising the step of administering a RET tyrosine kinase inhibitor to a patient in whom a cancer treatment with the RET tyrosine kinase inhibitor has been determined to be highly effective by the method according to claim 5.

8. A therapeutic agent for cancer, comprising a RET tyrosine kinase inhibitor as an active ingredient, wherein the therapeutic agent is to be administered to a patient in whom a cancer treatment with the RET tyrosine kinase inhibitor has been determined to be highly effective by the method according to claim 5.

9. A method for detecting the presence or absence in a sample of the polypeptide according to claim 2, the method comprising the steps of:
(a) contacting the sample with an agent intended for specifically detecting the presence or absence of the polypeptide in the sample; and
(b) detecting the presence or absence of the polypeptide.

10. An agent for detecting the presence or absence in a sample of a polypeptide in which CEP55 protein or part thereof and RET protein or part thereof are fused together for use in the method according to claim 9, the agent comprising an antibody as set forth below in (d):
(d) an antibody that binds to a polypeptide in which CEP55 protein and RET protein are fused together.

11. A method for determining the effectiveness of a cancer treatment with a RET tyrosine kinase inhibitor, the method comprising the step of detecting the presence or absence in a sample isolated from a patient of the polypeptide according to claim 2, wherein in a case where the presence of the polypeptide is detected, the cancer treatment with the RET inhibitor is determined to be highly effective in the patient.

12. An agent for determining the effectiveness of a cancer treatment with a RET inhibitor by the method according to claim 11, the agent comprising an antibody as set forth below in (d):
(d) an antibody that binds to a polypeptide in which CEP55 protein and RET protein are fused together.

13. A method for treatment of cancer, comprising the step of administering a RET tyrosine kinase inhibitor to a patient in whom a cancer treatment with the RET tyrosine kinase inhibitor has been determined to be highly effective by the method according to claim 11.

14. A therapeutic agent for cancer, comprising a RET tyrosine kinase inhibitor as an active ingredient, wherein the therapeutic agent is to be administered to a patient in whom a cancer treatment with the RET tyrosine kinase inhibitor has been determined to be highly effective by the method according to claim 11.


SEQUENCE LISTING (10 sequences)

SEQ ID NO: 1 - DNA, 2656 bp, Homo sapiens
  CDS (305)..(1699)
cacacctgatggtgtgactcggccgacgcgagcgccgcgcttcgcttcagctgctagctggcccaagggaggcgaccgcggagggtggcgaggggcggccaggacccgcagccccggggccgggccggtccggaccgccagggagggcaggtcagtgggcagatcgcgtccgcgggattcaatctctgcccgctctgataacagtccttttccctggcgctcacttcgtgcctggcacccggctgggcgcctcaagaccgttgtctcttcgatcgcttctttggacttggcgaccatttcagagatgtcttccagaagtaccaaagatttaattaaaagtaagtggggatcgaagcctagtaactccaaatccgaaactacattagaaaaattaaagggagaaattgcacacttaaagacatcagtggatgaaatcacaagtgggaaaggaaagctgactgataaagagagacacagacttttggagaaaattcgagtccttgaggctgagaaggagaagaatgcttatcaactcacagagaaggacaaagaaatacagcgactgagagaccaactgaaggccagatatagtactaccgcattgcttgaacagctggaagagacaacgagagaaggagaaaggagggagcaggtgttgaaagccttatctgaagagaaagacgtattgaaacaacagttgtctgctgcaacctcacgaattgctgaacttgaaagcaaaaccaatacactccgtttatcacagactgtggctccaaactgcttcaactcatcaataaataatattcatgaaatggaaatacagctgaaagatgctctggagaaaaatcagcagtggctcgtgtatgatcagcagcgggaagtctatgtaaaaggacttttagcaaagatctttgagttggaaaagaaaacggaaacagctgctcattcactcccacagcagacaaaaaagcctgaatcagaaggttatcttcaagaagagaagcagaaatgttacaacgatctcttggcaagtgcaaaaaaagatcttgaggttgaacgacaaaccataactcagctgagttttgaactgagtgaatttcgaagaaaatatgaagaaacccaaaaagaagttcacaatttaaatcagctgttgtattcacaaagaagggcagatgtgcaacatctggaagatgataggcataaaacagagaagatacaaaaactcagggaagagaatgatattgctaggggaaaacttgaagaagagaagaagagatccgaagagctcttatctcaggtccagtttctttacacatctctgctaaagcagcaagaagaacaaacaagggtagctctgttggaacaacagatgcaggcatgtactttagactttgaaaatgaaaaactcgaccgtcaacatgtgcagcatcaattgcatgtaattcttaaggagctccgaaaagcaagaaatcaaataacacagttggaatccttgaaacagcttcatgagtttgccatcacagagccattagtcactttccaaggagagactgaaaacagagaaaaagttgccgcctcaccaaaaagtcccactgctgcactcaatgaaagcctggtggaatgtcccaagtgcaatatacagtatccagccactgagcatcgcgatctgcttgtccatgtggaatactgttcaaagtagcaaaataagtatttgttttgatattaaaagattcaatactgtattttctgttagcttgtgggcattttgaattatatatttcacattttgcataaaactgcctatctacctttgacactccagcatgctagtgaatcatgtatcttttaggctgctgtgcatttctcttggcagtgatacctccctgacatggttcatcatcaggctgcaatgacagaatgtggtgagcagcgtctactgagactactaacattttgcactgtcaaaatacttggtgaggaaaagatagctcaggttattgctaatgggttaatgcaccagcaagcaaaatattttatgttttgggggttttgaaaaatcaaagataattaaccaaggatcttaactgtgttcgcattttttatccaagcacttagaaaacctacaatcctaattttgatgtccattgttaagaggtggtgatagatactattttttttttcatattgtatagcggttattagaaaagttggggattttcttgatctttattgctgcttaccattgaaacttaacccagctgtgttccccaactctgttctgcgcacgaaacagtatctgtttgaggcataatcttaagtggccacacacaatgttttctcttatgttatctggcagtaactgtaacttgaattacattagcacattctgcttagctaaaattgttaaaataaactttaataaacccatgtagccctctcatttgattgacagtattttagttatttttggcattcttaaagctgggcaatgtaatgatcagatctttgtttgtctgaacaggtatttttatacatgctttttgtaaaccaaaaacttttaaatttcttcaggttttctaacatgcttaccactgggctactgtaaatgagaaaagaataaaattatttaatgttttaaaaaaaaaaaaaaaaaaaa

SEQ ID NO: 2 - protein, 464 aa, Homo sapiens
MSSRSTKDLIKSKWGSKPSNSKSETTLEKLKGEIAHLKTSVDEITSGKGKLTDKERHRLLEKIRVLEAEKEKNAYQLTEKDKEIQRLRDQLKARYSTTALLEQLEETTREGERREQVLKALSEEKDVLKQQLSAATSRIAELESKTNTLRLSQTVAPNCFNSSINNIHEMEIQLKDALEKNQQWLVYDQQREVYVKGLLAKIFELEKKTETAAHSLPQQTKKPESEGYLQEEKQKCYNDLLASAKKDLEVERQTITQLSFELSEFRRKYEETQKEVHNLNQLLYSQRRADVQHLEDDRHKTEKIQKLREENDIARGKLEEEKKRSEELLSQVQFLYTSLLKQQEEQTRVALLEQQMQACTLDFENEKLDRQHVQHQLHVILKELRKARNQITQLESLKQLHEFAITEPLVTFQGETENREKVAASPKSPTAALNESLVECPKCNIQYPATEHRDLLVHVEYCSK

SEQ ID NO: 3 - DNA, 5629 bp, Homo sapiens
  CDS (191)..(3535)
agtcccgcgaccgaagcagggcgcgcagcagcgctgagtgccccggaacgtgcgtcgcgcccccagtgtccgtcgcgtccgccgcgccccgggcggggatggggcggccagactgagcgccgcacccgccatccagacccgccggccctagccgcagtccctccagccgtggccccagcgcgcacgggcgatggcgaaggcgacgtccggtgccgcggggctgcgtctgctgttgctgctgctgctgccgctgctaggcaaagtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggcatgtcagacccgaactggcctggagagagtcctgtaccactcacgagagctgatggcactaacactgggtttccaagatatccaaatgatagtgtatatgctaactggatgctttcaccctcagcggcaaaattaatggacacgtttgatagttaacatttctttgtgaaaggtaatggactcacaaggggaagaaacatgctgagaatggaaagtctaccggccctttctttgtgaacgtcacattggccgagccgtgttcagttcccaggtggcagactcgtttttggtagtttgttttaacttccaaggtggttttacttctgatagccggtgattttccctcctagcagacatgccacaccgggtaagagctctgagtcttagtggttaagcattcctttctcttcagtgcccagcagcacccagtgttggtctgtgtccatcagtgaccaccaacattctgtgttcacatgtgtgggtccaacacttactacctggtgtatgaaattggacctgaactgttggatttttctagttgccgccaaacaaggcaaaaaaatttaaacatgaagcacacacacaaaaaaggcagtaggaaaaatgctggccctgatgacctgtccttattcagaatgagagactgcggggggggcctgggggtagtgtcaatgcccctccagggctggaggggaagaggggccccgaggatgggcctgggctcagcattcgagatcttgagaatgatttttttttaatcatgcaacctttccttaggaagacatttggttttcatcatgattaagatgattcctagatttagcacaatggagagattccatgccatctttactatgtggatggtggtatcagggaagagggctcacaagacacatttgtcccccgggcccaccacatcatcctcacgtgttcggtactgagcagccactacccctgatgagaacagtatgaagaaagggggctgttggagtcccagaattgctgacagcagaggctttgctgctgtgaatcccacctgccaccagcctgcagcacaccccacagccaagtagaggcgaaagcagtggctcatcctacctgttaggagcaggtagggcttgtactcactttaatttgaatcttatcaacttactcataaagggacaggctagctagctgtgttagaagtagcaatgacaatgaccaaggactgctacacctctgattacaattctgatgtgaaaaagatggtgtttggctcttatagagcctgtgtgaaaggcccatggatcagctcttcctgtgtttgtaatttaatgctgctacaagatgtttctgtttcttagattctgaccatgactcataagcttcttgtcattcttcattgcttgtttgtggtcacagatgcacaacactcctccagtcttgtgggggcagcttttgggaagtctcagcagctcttctggctgtgttgtcagcactgtaacttcgcagaaaagagtcggattaccaaaacactgcctgctcttcagacttaaagcactgataggacttaaaatagtctcattcaaatactgtattttatataggcatttcacaaaaacagcaaaattgtggcattttgtgaggccaaggcttggatgcgtgtgtaatagagccttgtggtgtgtgcgcacacacccagagggagagtttgaaaaatgcttattggacacgtaacctggctctaatttgggctgtttttcagatacactgtgataagttcttttacaaatatctatagacatggtaaacttttggttttcagatatgcttaatgatagtcttactaaatgcagaaataagaataaactttctcaaattattaaaaatgcctacacagtaagtgtgaattgctgcaacaggtttgttctcaggagggtaagaactccaggtctaaacagctgacccagtgatggggaatttatccttgaccaatttatccttgaccaataacctaattgtctattcctgagttataaaagtccccatccttattagctctactggaattttcatacacgtaaatgcagaagttactaagtattaagtattactgagtattaagtagtaatctgtcagttattaaaatttgtaaaatctatttatgaaaggtcattaaaccagatcatgttcctttttttgtaatcaaggtgactaagaaaatcagttgtgtaaataaaatcatgtatcataaaaaaaaaaaaaaaa

SEQ ID NO: 4 - protein, 1114 aa, Homo sapiens
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 5 - DNA, 4174 bp, Homo sapiens
  CDS (191)..(3409)
agtcccgcgaccgaagcagggcgcgcagcagcgctgagtgccccggaacgtgcgtcgcgcccccagtgtccgtcgcgtccgccgcgccccgggcggggatggggcggccagactgagcgccgcacccgccatccagacccgccggccctagccgcagtccctccagccgtggccccagcgcgcacgggcgatggcgaaggcgacgtccggtgccgcggggctgcgtctgctgttgctgctgctgctgccgctgctaggcaaagtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctagcaccgctgtcccctctgcactatccttcctctctgtgatgctttttaaaaatgtttctggtctgaacaaaaccaaagtctgctctgaacctttttatttgtaaatgtctgactttgcatccagtttacatttaggcattattgcaactatgtttttctaaaaggaagtgaaaataagtgtaattaccacattgcccagcaacttaggatggtagaggaaaaaacagatcagggcggaactctcaggggagaccaagaacaggttgaataaggcgcttctggggtgggaatcaagtcatagtacttctactttaactaagtggataaatatacaaatctggggaggtattcagttgagaaaggagccaccagcaccactcagcctgcactgggagcacagccaggttcccccagacccctcctgggcaggcaggtgcctctcagaggccacccggcactggcgagcagccactggccaagcctcagccccagtcccagccacatgtcctccatcaggggtagcgaggttgcaggagctggctggccctgggaggacgcacccccactgctgttttcacatcctttcccttacccaccttcaggacggttgtcacttatgaagtcagtgctaaagctggagcagttgctttttgaaagaacatggtctgtggtgctgtggtcttacaatggacagtaaatatggttcttgccaaaactccttcttttgtctttgattaaatactagaaatttaaaaaaaaaaaaaaa

SEQ ID NO: 6 - protein, 1072 aa, Homo sapiens
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 7 - DNA, 4066 bp, Homo sapiens
  CDS (305)..(1972)
cacacctgatggtgtgactcggccgacgcgagcgccgcgcttcgcttcagctgctagctggcccaagggaggcgaccgcggagggtggcgaggggcggccaggacccgcagccccggggccgggccggtccggaccgccagggagggcaggtcagtgggcagatcgcgtccgcgggattcaatctctgcccgctctgataacagtccttttccctggcgctcacttcgtgcctggcacccggctgggcgcctcaagaccgttgtctcttcgatcgcttctttggacttggcgaccatttcagagatgtcttccagaagtaccaaagatttaattaaaagtaagtggggatcgaagcctagtaactccaaatccgaaactacattagaaaaattaaagggagaaattgcacacttaaagacatcagtggatgaaatcacaagtgggaaaggaaagctgactgataaagagagacacagacttttggagaaaattcgagtccttgaggctgagaaggagaagaatgcttatcaactcacagagaaggacaaagaaatacagcgactgagagaccaactgaaggccagatatagtactaccgcattgcttgaacagctggaagagacaacgagagaaggagaaaggagggagcaggtgttgaaagccttatctgaagagaaagacgtattgaaacaacagttgtctgctgcaacctcacgaattgctgaacttgaaagcaaaaccaatacactccgtttatcacaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggcatgtcagacccgaactggcctggagagagtcctgtaccactcacgagagctgatggcactaacactgggtttccaagatatccaaatgatagtgtatatgctaactggatgctttcaccctcagcggcaaaattaatggacacgtttgatagttaacatttctttgtgaaaggtaatggactcacaaggggaagaaacatgctgagaatggaaagtctaccggccctttctttgtgaacgtcacattggccgagccgtgttcagttcccaggtggcagactcgtttttggtagtttgttttaacttccaaggtggttttacttctgatagccggtgattttccctcctagcagacatgccacaccgggtaagagctctgagtcttagtggttaagcattcctttctcttcagtgcccagcagcacccagtgttggtctgtgtccatcagtgaccaccaacattctgtgttcacatgtgtgggtccaacacttactacctggtgtatgaaattggacctgaactgttggatttttctagttgccgccaaacaaggcaaaaaaatttaaacatgaagcacacacacaaaaaaggcagtaggaaaaatgctggccctgatgacctgtccttattcagaatgagagactgcggggggggcctgggggtagtgtcaatgcccctccagggctggaggggaagaggggccccgaggatgggcctgggctcagcattcgagatcttgagaatgatttttttttaatcatgcaacctttccttaggaagacatttggttttcatcatgattaagatgattcctagatttagcacaatggagagattccatgccatctttactatgtggatggtggtatcagggaagagggctcacaagacacatttgtcccccgggcccaccacatcatcctcacgtgttcggtactgagcagccactacccctgatgagaacagtatgaagaaagggggctgttggagtcccagaattgctgacagcagaggctttgctgctgtgaatcccacctgccaccagcctgcagcacaccccacagccaagtagaggcgaaagcagtggctcatcctacctgttaggagcaggtagggcttgtactcactttaatttgaatcttatcaacttactcataaagggacaggctagctagctgtgttagaagtagcaatgacaatgaccaaggactgctacacctctgattacaattctgatgtgaaaaagatggtgtttggctcttatagagcctgtgtgaaaggcccatggatcagctcttcctgtgtttgtaatttaatgctgctacaagatgtttctgtttcttagattctgaccatgactcataagcttcttgtcattcttcattgcttgtttgtggtcacagatgcacaacactcctccagtcttgtgggggcagcttttgggaagtctcagcagctcttctggctgtgttgtcagcactgtaacttcgcagaaaagagtcggattaccaaaacactgcctgctcttcagacttaaagcactgataggacttaaaatagtctcattcaaatactgtattttatataggcatttcacaaaaacagcaaaattgtggcattttgtgaggccaaggcttggatgcgtgtgtaatagagccttgtggtgtgtgcgcacacacccagagggagagtttgaaaaatgcttattggacacgtaacctggctctaatttgggctgtttttcagatacactgtgataagttcttttacaaatatctatagacatggtaaacttttggttttcagatatgcttaatgatagtcttactaaatgcagaaataagaataaactttctcaaattattaaaaatgcctacacagtaagtgtgaattgctgcaacaggtttgttctcaggagggtaagaactccaggtctaaacagctgacccagtgatggggaatttatccttgaccaatttatccttgaccaataacctaattgtctattcctgagttataaaagtccccatccttattagctctactggaattttcatacacgtaaatgcagaagttactaagtattaagtattactgagtattaagtagtaatctgtcagttattaaaatttgtaaaatctatttatgaaaggtcattaaaccagatcatgttcctttttttgtaatcaaggtgactaagaaaatcagttgtgtaaataaaatcatgtatcataaaaaaaaaaaaaaaa

SEQ ID NO: 8 - protein, 555 aa, Homo sapiens
MSSRSTKDLIKSKWGSKPSNSKSETTLEKLKGEIAHLKTSVDEITSGKGKLTDKERHRLLEKIRVLEAEKEKNAYQLTEKDKEIQRLRDQLKARYSTTALLEQLEETTREGERREQVLKALSEEKDVLKQQLSAATSRIAELESKTNTLRLSQEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 9 - DNA, 2611 bp, Homo sapiens
  CDS (305)..(1846)
cacacctgatggtgtgactcggccgacgcgagcgccgcgcttcgcttcagctgctagctggcccaagggaggcgaccgcggagggtggcgaggggcggccaggacccgcagccccggggccgggccggtccggaccgccagggagggcaggtcagtgggcagatcgcgtccgcgggattcaatctctgcccgctctgataacagtccttttccctggcgctcacttcgtgcctggcacccggctgggcgcctcaagaccgttgtctcttcgatcgcttctttggacttggcgaccatttcagagatgtcttccagaagtaccaaagatttaattaaaagtaagtggggatcgaagcctagtaactccaaatccgaaactacattagaaaaattaaagggagaaattgcacacttaaagacatcagtggatgaaatcacaagtgggaaaggaaagctgactgataaagagagacacagacttttggagaaaattcgagtccttgaggctgagaaggagaagaatgcttatcaactcacagagaaggacaaagaaatacagcgactgagagaccaactgaaggccagatatagtactaccgcattgcttgaacagctggaagagacaacgagagaaggagaaaggagggagcaggtgttgaaagccttatctgaagagaaagacgtattgaaacaacagttgtctgctgcaacctcacgaattgctgaacttgaaagcaaaaccaatacactccgtttatcacaggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctagcaccgctgtcccctctgcactatccttcctctctgtgatgctttttaaaaatgtttctggtctgaacaaaaccaaagtctgctctgaacctttttatttgtaaatgtctgactttgcatccagtttacatttaggcattattgcaactatgtttttctaaaaggaagtgaaaataagtgtaattaccacattgcccagcaacttaggatggtagaggaaaaaacagatcagggcggaactctcaggggagaccaagaacaggttgaataaggcgcttctggggtgggaatcaagtcatagtacttctactttaactaagtggataaatatacaaatctggggaggtattcagttgagaaaggagccaccagcaccactcagcctgcactgggagcacagccaggttcccccagacccctcctgggcaggcaggtgcctctcagaggccacccggcactggcgagcagccactggccaagcctcagccccagtcccagccacatgtcctccatcaggggtagcgaggttgcaggagctggctggccctgggaggacgcacccccactgctgttttcacatcctttcccttacccaccttcaggacggttgtcacttatgaagtcagtgctaaagctggagcagttgctttttgaaagaacatggtctgtggtgctgtggtcttacaatggacagtaaatatggttcttgccaaaactccttcttttgtctttgattaaatactagaaatttaaaaaaaaaaaaaaa

SEQ ID NO: 10 - protein, 513 aa, Homo sapiens
MSSRSTKDLIKSKWGSKPSNSKSETTLEKLKGEIAHLKTSVDEITSGKGKLTDKERHRLLEKIRVLEAEKEKNAYQLTEKDKEIQRLRDQLKARYSTTALLEQLEETTREGERREQVLKALSEEKDVLKQQLSAATSRIAELESKTNTLRLSQEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF